(19) United States
(12) Patent Application Publication
(10) Pub. No.: US 2025/0152042 A1
(43) Pub. Date: May 15, 2025

(54) WEARABLE MUSCLE ACTIVITY MEASUREMENT DEVICE FOR DIAGNOSING MUSCLE CONDITION

(71) Applicant: ELECTRONICS AND TELECOMMUNICATIONS RESEARCH INSTITUTE, Daejeon (KR)

(72) Inventors: YunJeong KIM, Daejeon (KR); Hye Jin KIM, Daejeon (KR); Hanbit JIN, Daejeon (KR)

(21) Appl. No.: 18/937,546

(22) Filed: Nov. 5, 2024

(30) Foreign Application Priority Data
Nov. 10, 2023 (KR) 10-2023-0155773
Aug. 30, 2024 (KR) 10-2024-0117460

(51) Int. Cl. A61B 5/11; A61B 5/00

(52) U.S. Cl. CPC A61B 5/1107; A61B 5/4519

(57) ABSTRACT

Provided is a wearable muscle activity measurement device for diagnosing a muscle condition, and a muscle activity measurement method thereof. The device includes a main substrate, a communication module connected to the main substrate, air pockets provided on one side of the main substrate adjacent to the communication module, a motion sensor provided on the other side of the main substrate opposing the air pockets, mechanomyography sensors provided on the center of the main substrate between the motion sensor and the air pockets to detect vibration signals; and a force myography sensor provided on the main substrate between the mechanomyography sensors to detect pressure signals.

### CROSS-REFERENCE TO RELATED APPLICATIONS

This U.S. non-provisional patent application claims priority under 35 U.S.C. § 119 of Korean Patent Application No. 10-2023-0155773, filed on Nov. 10, 2023, and No. 10-2024-0117460, filed on Aug. 30, 2024, the entire contents of which are hereby incorporated by reference.

### BACKGROUND

The present disclosure herein relates to a muscle activity measurement device, and more particularly, to a wearable muscle activity measurement device for diagnosing a muscle condition.

Typical electromyogram methods require electrical contact with the skin, so that there is a problem in that signals are distorted due to the influence of sweat, movement, degree of skin adhesion, change in ion concentration on the body surface or in the body. In addition, the skin may feel irritated due to the attachment of an electrode. Accordingly, a typical muscle condition monitoring system based only on electromyogram (EMG) has a limitation in that accurate muscle activity analysis is limited depending on the situation.

### SUMMARY

The present disclosure provides a wearable muscle activity measurement device capable of minimizing noise.

An embodiment of the inventive concept provides a wearable muscle activity measurement device for diagnosing a muscle condition. The device includes a main substrate, a communication module connected to the main substrate, air pockets provided on one side of the main substrate adjacent to the communication module, a motion sensor provided on the other side of the main substrate opposing the air pockets, mechanomyography sensors provided on the center of the main substrate between the motion sensor and the air pockets to detect vibration signals; and a force myography sensor provided on the main substrate between the mechanomyography sensors to detect pressure signals.

In an embodiment, the communication module may include a module substrate, and a control unit provided on the module substrate and connected to the motion sensor, the mechanomyography sensors, and the force myography sensor. In an embodiment, the control unit may overlap a first peak region of the vibration signals and second peak region of the pressure signals to distinguish noise regions of the pressure signals.

In an embodiment, the control unit may use a first peak value of first central regions between first edge regions of the vibration signals to calculate muscle activity.

In an embodiment, the control unit may use a second peak value of the pressure signals having a second central region overlapping the first central region to calculate muscle fatigue.

In an embodiment, the device may further include a pressure sensor provided on the module substrate or the air pockets.

In an embodiment, the device may further include a pressure sensor provided on the air pockets or the module substrate.

In an embodiment, the device may further include an air hose connecting the pressure sensor with the air pocket, which are on the module substrate.

In an embodiment, the force myography sensor may include a first sensor substrate, a plurality of first electrodes on the first sensor substrate, a ring spacer provided on an edge of the first sensor substrate, which is an outer periphery of the first electrodes, a pressure-sensitive structure provided on the ring spacer and the first electrodes, and a passivation film provided on the pressure-sensitive structure.

In an embodiment, each of the above mechanomyography sensors may include a second sensor substrate, a housing case provided on an edge of the second sensor substrate, second electrodes provided on the center of the second sensor substrate in the housing case, a piezoelectric composite provided on the second electrodes, a vibration transmission structure on the piezoelectric composite, an elastic spring on the vibration transmission structure, and a vibration transmission plate on the elastic spring.

In an embodiment, the main substrate may include a textile substrate.

In an embodiment of the inventive concept, a method for measuring muscle activity includes obtaining vibration signals and pressure signals, using the vibration signals to calculate muscle activity, using the pressure signals to calculate power spectra, and using the power spectra to calculate muscle fatigue.

In an embodiment, the vibration signals may include a first vibration signal, a tenth vibration signal, and a twentieth vibration signal, which have first peak values in a first peak region, wherein the first peak region may include a first central region and first edge regions on both sides of the first central region.

In an embodiment, the pressure signals may include a first pressure signal, a tenth pressure signal, and a twentieth pressure signal, which have second peak values in a second peak region, wherein the second peak region may include a second central region overlapping the first central region, second edge regions overlapping the first edge regions, and a noise region at an outer periphery of the second edge regions.

In an embodiment, the power spectra may include a first power spectrum, a tenth power spectrum, and a twentieth power spectrum, which are respectively obtained by Fourier transform of the first pressure signal, the tenth pressure signal, and the twentieth pressure signal.

In an embodiment, the method further include using the first power spectrum and the twentieth power spectrum to obtain a first cumulative power having a first spectral edge frequency 30 value and a twentieth cumulative power having a second spectral edge frequency 30 value, wherein the muscle fatigue may be calculated as a percentage of a value obtained by dividing a difference between the first spectral edge frequency 30 value and the second spectral edge frequency 30 value by the first spectral edge frequency 30 value.

In an embodiment of the inventive concept, a method for measuring muscle activity includes obtaining vibration signals including a first vibration signal, a tenth vibration signal, and a twentieth vibration signal overlapping in a first peak region, and pressure signals including a first pressure signal, a tenth pressure signal, and a twentieth pressure signal overlapping in second peak region, integrating first peak values of the first vibration signal, the tenth vibration signal, and the twentieth vibration signal in a first central region of the first peak region to calculate muscle activity, transforming the first pressure signal, the tenth pressure signal, and the twentieth pressure signal in a second central region of the second peak region by a Fourier transform method to calculate power spectra including a first power spectrum, a tenth power spectrum, and a twentieth power spectrum, first cumulative power having a first spectral edge frequency 30 value and a twentieth cumulative power having a second spectral edge frequency 30 value, and calculating a difference between the first spectral edge frequency 30 value and the second spectral edge frequency 30 value as muscle fatigue corresponding to a distribution rate of the first spectral edge frequency 30 value.

In an embodiment, the first central region and the second central region may overlap each other.

In an embodiment, the first peak region may further include a first edge region provided on both sides of the first central region, and the second peak area may further include a second edge region provided on both sides of the second central region and overlapping the first edge region.

In an embodiment, the second peak region may further include a noise region at an outer periphery of the second edge region.

In an embodiment, the calculating of the power spectra may include removing the second edge regions, and the first pressure signal, the tenth pressure signal, and the twentieth pressure signal in the noise region.

### BRIEF DESCRIPTION OF THE FIGURES

The accompanying drawings are included to provide a further understanding of the inventive concept, and are incorporated in and constitute a part of this specification. The drawings illustrate embodiments of the inventive concept and, together with the description, serve to explain principles of the inventive concept. In the drawings:

FIG. 1 and FIG. 2 are plan views showing an example of a wearable muscle activity measurement device for diagnosing a muscle condition according to an embodiment of the inventive concept;

FIG. 3 is a cross-sectional view showing an example of a mechanomyography sensor of FIG. 1 and FIG. 2;

FIG. 4 is a cross-sectional view showing an example of a force myography sensor of FIG. 1 and FIG. 2;

FIG. 5 shows graphs showing an example of vibration signals and pressure signals of the mechanomyography sensors and the force myography sensor of FIG. 1 and FIG. 2;

FIG. 6 shows graphs showing an example of power spectra obtained from the pressure signals of FIG. 5;

FIG. 7 shows graphs showing an example of a first cumulative power and a twentieth cumulative power of the power spectra of FIG. 6;

FIG. 8 are images showing an example of a wearable muscle activity measurement device for diagnosing a muscle condition according to an embodiment of the inventive concept;

FIGS. 9A, 9B, and 9C are views showing an example of a textile transfer process of a wearable muscle activity measurement device for diagnosing a muscle condition of the inventive concept;

FIG. 10 is a plan view showing an example of an array of the force myography sensor and the mechanomyography sensors of FIG. 1 and FIG. 2, and a motion sensor;

FIG. 11 is an image showing an illustration of wearing a wearable muscle activity measurement device for diagnosing a muscle condition of the inventive concept;

FIG. 12 is a view showing an example of a display device for displaying muscle activity statistical data measured from a wearable muscle activity measurement device for diagnosing a muscle condition of the inventive concept; and

FIG. 13 is a flowchart showing an example of a method for measuring muscle activity of a wearable muscle activity measurement device for diagnosing a muscle condition according to an embodiment of the inventive concept.

### DETAILED DESCRIPTION

In order to facilitate sufficient understanding of the configuration and effects of a technical idea of the present invention, preferred embodiments of the technical idea of the present invention will be described with reference to the accompanying drawings. However, the technical idea of the present invention is not limited to the embodiments set forth below, and may be embodied in various forms and modified in many alternate forms. Rather, the present embodiments are provided such that the disclosure of the technical idea of the present invention will be complete, and to fully convey the scope of the present invention to those skilled in the art to which the present invention pertains.

Like reference numerals refer to like elements throughout the specification. The embodiments described in the present specification will be described with reference to plan views, perspective views, and/or cross-sectional views, which are ideal illustrations of the technical idea of the present invention. In the drawings, the thickness of regions are exaggerated for an effective description of technical contents. Thus, the regions illustrated in the drawings have schematic properties, and the shapes of the regions illustrated in the drawings are intended to exemplify specific shapes of regions of a device and are not intended to limit the scope of the present invention. Although various terms are used in various embodiments of the present specification to describe various elements, these elements should not be limited by these terms. These terms are only used to distinguish one element from another. The embodiments described and illustrated herein also include complementary embodiments thereof.

The terms used herein are for the purpose of describing embodiments and are not intended to be limiting of the present invention. In the present specification, singular forms include plural forms unless the context clearly indicates otherwise. As used herein, the terms ‘comprises’ and/or ‘comprising’ are intended to be inclusive of the stated elements, and do not exclude the possibility of the presence or the addition of one or more other elements.

Hereinafter, the preferred embodiments of the technical idea of the present invention will be described with reference to the accompanying drawings to describe the present invention in detail.

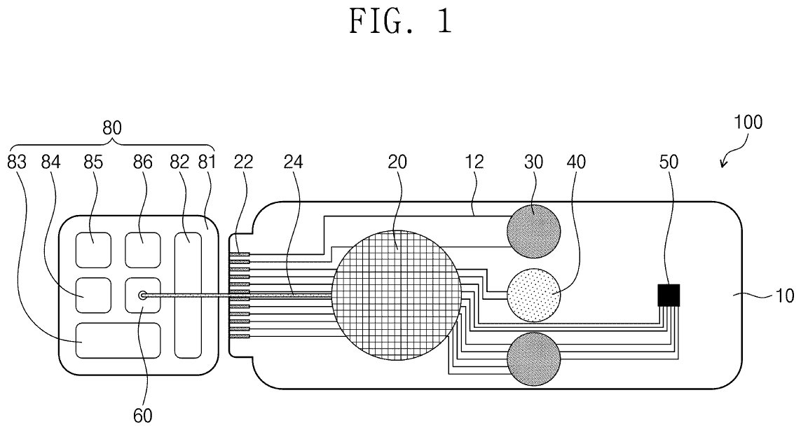

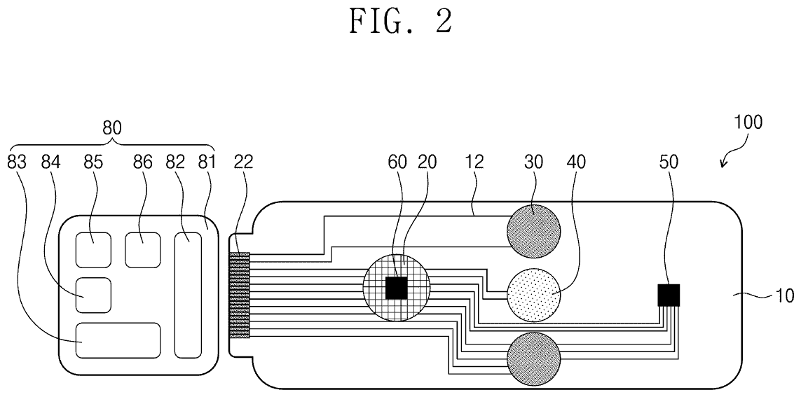

FIG. 1 and FIG. 2 show an example of a wearable muscle activity measurement device 100 for diagnosing a muscle condition according to an embodiment of inventive concept.

Referring to FIG. 1 and FIG. 2, the wearable muscle activity measurement device 100 for diagnosing a muscle condition of the present invention may include a main substrate 10, a communication module 80, air pockets 20, a mechanomyography (MMG) sensors 30, a force mammography (FMG) sensor 40, a motion sensor 50, and a pressure sensor 60.

The main substrate 10 may support the air pockets 20, the mechanomyography sensors 30, the force myography sensor 40, and the motion sensor 50. For example, the main substrate 10 may include a stretchable substrate such as rubber. Alternatively, the main board 10 may include a flexible substrate such as a textile, but the embodiment of the inventive concept is not limited thereto. The main substrate 10 may have a contact pad 22.

The communication module 80 may be connected to one side of the main substrate 10. The communication module 80 may obtain sensing signals of the mechanomyography sensors 30, the force myography sensor 40, the motion sensor 50, and the pressure sensor 60 and output the sensing signals in a wired or wireless manner. According to an embodiment, the communication module 80 may include a module substrate 81, a connector 82, a power unit 83, a control unit 84, a communication unit 85, and a signal processing unit 86.

The module substrate 81 may include a printed circuit board. The connector 82 may be provided adjacent to the contact pad 22. The connector 82 may include an arm socket for accommodating the contact pad 22 of the main substrate 10. The power unit 83 may be provided on the module substrate 81 adjacent to the connector 82. The power unit 83 may provide a power voltage to the control unit 84, the communication unit 85, and the signal processing unit 86. The power unit 83 may include a battery. The control unit 84 may obtain muscle activity of the human body by using detection signals of the mechanomyography sensors 30, the force myography sensor 40, the motion sensor 50, and the pressure sensor 60. The control unit 84 may output information on the muscle activity to the outside through the communication unit 85. The communication unit 85 may output the information on the muscle activity in a wired or wireless manner. The communication unit 85 may include a transmitter, a receiver, and an antenna. The signal processing unit 86 may drive and process signals of the mechanomyography sensors 30, the force myography sensor 40, the motion sensor 50, and the pressure sensor 60. The signal processing unit 86 may include an analog-to-digital (AD) converter.

The pressure sensor 60 may be provided on the module substrate 81 or may be provided on the air pockets 20 of the main substrate 10. The pressure sensor 60 may detect a pressure between the main substrate 10 and the human body. The pressure sensor 60 may function or be used as a relative pressure sensor or absolute pressure sensor depending on the position thereof.

Referring to FIG. 1, the pressure sensor 60 may be mounted on the module substrate 81 of the communication module 80. The pressure sensor 60 may be connected to the air pockets 20 on the main substrate 10 by means of the air hose 24. The pressure sensor 60 may include a differential pressure sensor. The pressure sensor 60 may detect a change in pressure in the human body by using a difference between the pressure of the air pockets 20 and the atmospheric pressure.

Referring to FIG. 2, the pressure sensor 60 may be mounted on the air pockets 20. The pressure sensor 60 may include an absolute pressure sensor. The pressure sensor 60 may directly detect the change in pressure in the human body.

Referring to FIG. 1 and FIG. 2, the air pockets 20 may be provided on one side of the main substrate 10 adjacent to the communication module 80. For example, the air pockets 20 may include an embossing film or a polymer having a pressure higher than atmospheric pressure. Alternatively, the air pockets 20 may include an air cap, but the embodiment of the inventive concept is not limited thereto.

The motion sensor 50 may be provided on the other side of main the substrate 10. The motion sensor 50 may sense a motion of the human body. The motion sensor 50 may include an optical sensor. Alternatively, the motion sensor 50 may include a voltage sensor or a resistance sensor, but the embodiment of the present invention is not limited thereto.

The mechanomyography sensors 30 may be provided on the center of the main substrate 10 between the air pockets 20 and the motion sensor 50. The mechanomyography sensors 30 may detect a low-frequency vibration of the human body. For example, the mechanomyography sensors 30 may include a piezoelectric (PZT) sensor, but the embodiment of the inventive concept is not limited thereto.

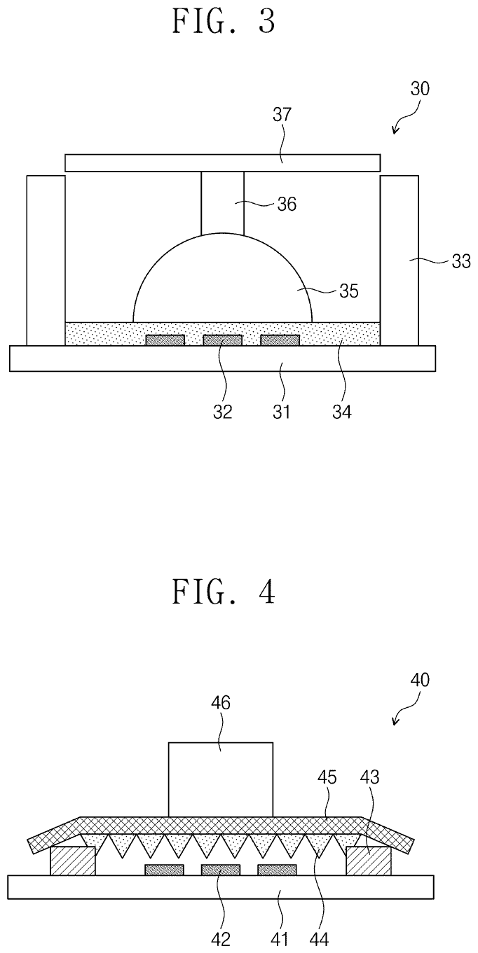

FIG. 3 shows an example of the mechanomyography sensors 30 of FIG. 1 and FIG. 2.

Referring to FIG. 3, the mechanomyography sensors 30 may include a first sensor substrate 31, first electrodes 32, a housing case 33, a piezoelectric composite 34, a vibration transmission structure 35, an elastic spring 36, and a vibration transmission plate 37. The first sensor substrate 31 may support or mount the first electrodes 32, the housing case 33, the piezoelectric composite 34, the vibration transmission structure 35, the elastic spring 36, and the vibration transmission plate 37. The first electrodes 32 may be provided on the center of the first sensor substrate 31. The housing case 33 may be disposed on an edge of the first sensor substrate 31 to surround the first electrodes 32. The piezoelectric composite 34 may be provided on the first electrodes 32 and the first sensor substrate 31 in the housing case 33. The piezoelectric composite 34 may include a piezoelectric (PZT) body. The vibration transmission structure 35 may be provided on the center of the piezoelectric composite 34. The vibration transmission structure 35 may have a hemispherical shape or a dome shape. The elastic spring 36 may be provided on the center of the vibration transmission structure 35. The vibration transmission plate 37 may be provided on the elastic spring 36. When external vibration is applied to the vibration transmission plate 37, the vibration transmission plate 37, the elastic spring 36, and the vibration transmission structure 35 may move in a vertical direction. The piezoelectric composite 34 may generate an electrical mechanomyography signal according to the movement of the vibration transmission plate 37, the elastic spring 36, and the vibration transmission structure 35. The first electrodes 32 may transmit the mechanomyography signal to the control unit 84.

Referring back to FIG. 1 and FIG. 2, the force myography sensor 40 may be provided on the main substrate 10 between the mechanomyography sensors 30. The force myography sensor 40 may detect a pressure of the human body. For example, the force myography sensor 40 may include a resistance sensor or a piezoresistive sensor, but the embodiment of the inventive concept is not limited thereto.

FIG. 4 shows an example of the force myography sensor 40 of FIG. 1 and FIG. 2.

Referring to FIG. 4, the force myography sensor 40 may include a second sensor substrate 41, second electrodes 42, a ring spacer 43, a pressure-sensitive structure 44, a passivation film 45, and a pressure concentration structure 46. The second sensor substrate 41 may support or mount the second electrodes 42, the ring spacer 43, the pressure-sensitive structure 44, the passivation film 45, and the pressure concentration structure 46. The second electrodes 42 may be provided on the center of the second sensor substrate 41. The ring spacer 43 may be provided on an edge of the second sensor substrate 41 at an outer periphery of the second electrodes 42. The ring spacer 43 may include an air vent which allows air to flow in and out. Although not illustrated, the ring spacer 43 may have an open structure like a C-shape. The pressure-sensitive structure 44 may be supported on the ring spacer 43. The pressure-sensitive structure 44 may be provided in an upper portion of the second electrodes 42. The pressure-sensitive structure 44 may be separated from the second electrodes 42. The pressure-sensitive structure 44 may have a sawtooth shape when viewed in a vertical direction. The passivation film 45 may be provided on the pressure-sensitive structure 44 and the ring spacer 43. The pressure concentration structure 46 may be provided on the center of the passivation film 45. If the pressure concentration structure 46, the passivation film 45, and the pressure concentration structure 44 are lowered by an external load, the second electrodes 42 may come in contact with the pressure concentration structure 44, and may generate a voltage or current signal to output the voltage or current signal to the control unit 84. The voltage or current signal may be generated by a change in resistance change according to the area of the contact between the second electrodes 42 and the pressure concentration structure 44. That is, the second electrodes 42 and the pressure concentration structure 44 may generate a voltage or current signal in a resistive manner. Alternatively, the second electrodes 42 and the pressure-sensitive structure 44 may generate a voltage or current signal in a capacitive manner, but the embodiment of the inventive concept is not limited thereto.

Referring back to FIG. 1 and FIG. 2, the mechanomyography sensors 30, the force myography sensor 40, the motion sensor 50, and the pressure sensor 60 may be connected to the communication module 80 by means of electrode lines 12. The communication module 80 may obtain muscle activity of the human body by using a detection signal of the mechanomyography sensors 30, the force myography sensor 40, the motion sensor 50, and the pressure sensor 60. Alternatively, the communication module 80 may provide the detection signal to an external computer or server to obtain muscle activity, but the embodiment of the inventive concept is not limited thereto.

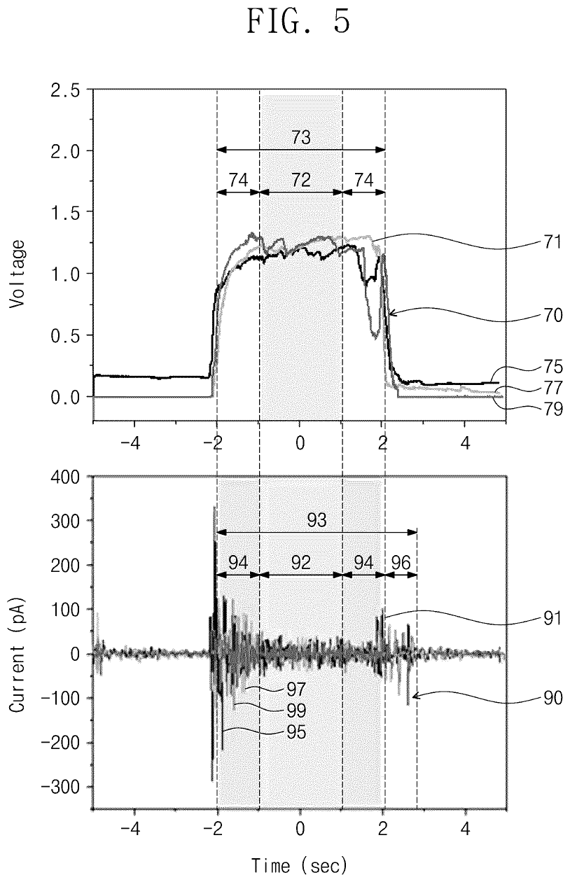

FIG. 5 shows an example of vibration signals 70 and pressure signals 90 of the mechanomyography sensors 30 and the force myography sensor 40 of FIG. 1 and FIG. 2.

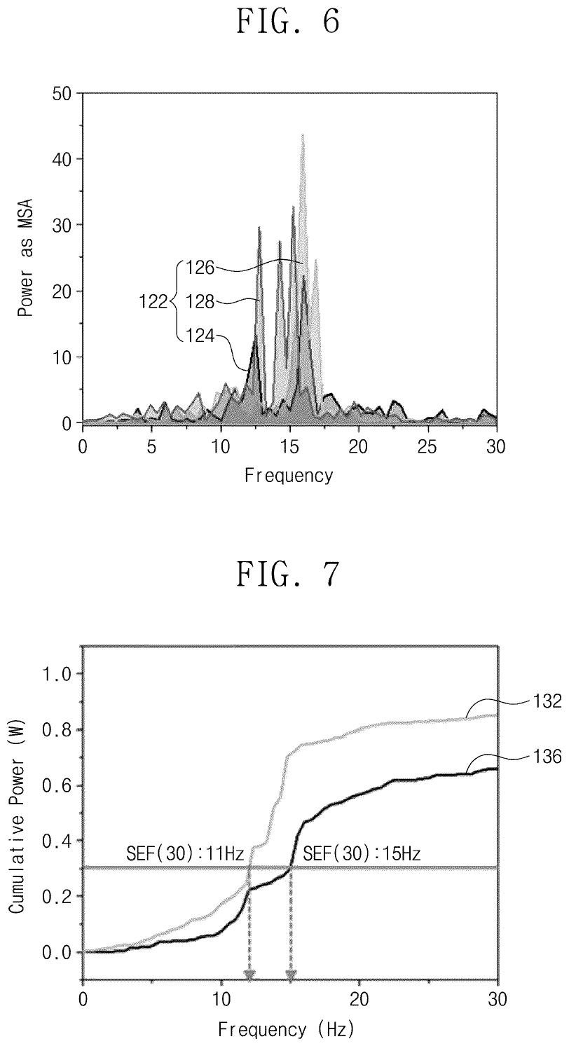

Referring to FIG. 1, FIG. 2, FIG. 5, and FIG. 6, the control unit 84 may obtain the vibration signals 70 and the pressure signals 90 from the mechanomyography sensors 30 and the force myography sensor 40. The vibration signals 70 and the pressure signals 90 may each include a voltage value and a current value over time. The vibration signals 70 may include a first vibration signal 75, a tenth vibration signal 57, and a twentieth vibration signal 79. The first vibration signal 75 may be obtained at the beginning of exercise of the human body, the tenth vibration signal 57 may be obtained in the middle of the exercise, and the twentieth vibration signal 79 may be obtained at the end of the exercise about 20 minutes later. The first vibration signal 75, the tenth vibration signal 57, and the twentieth vibration signal 79 may overlap in a first peak region 73 of about 4 seconds. The first peak region 73 may include a first central region 72 and first edge regions 74. The first central region 72 may be disposed between the first edge regions 74. The first central region 72 is a time region of about 2 seconds, and each of the first edge regions 74 may be a time region of about one second at an outer periphery of the first central region 72. The control unit 84 may obtain muscle activity by integrating first peak values 71 of the first central region 72 of the vibration signals 70.

The pressure signals 90 may include a first pressure signal 95, a tenth pressure signal 97, and a twentieth pressure signal 99. The first pressure signal 95 may be obtained simultaneously with the first vibration signal 75 at the beginning of the exercise of the human body, a second pressure signal may be obtained simultaneously with a second vibration signal in the middle of the exercise, and a third pressure signal may be obtained simultaneously with a third vibration signal at the end of the exercise. The first pressure signal 95, the tenth pressure signal 97, and the twentieth pressure signal 99 may overlap in a second peak region 93. The second peak region 93 may include a second central region 92, second edge regions 94, and a noise region 96. The second central region 92 may be a time region of about 2 seconds, which is the same as the first central region 72. The second edge regions 94 may be disposed on both sides of the second central region 92 in the same manner as the first edge region 74. Each of the second edge regions 94 may be a time region of about one second. The noise region 96 may be provided at an outer periphery of the second edge regions 94.

The control unit 84 may overlap the first peak region 73 of the vibration signals 70 and the second peak region 93 of the pressure signals 90 to remove a second peak value 91 of input signals of the second edge regions 94 and the noise region 96, and may obtain the second peak value 91 of the pressure signals 90 in the second central region 92.

FIG. 6 shows an example of power spectra 122 obtained from the vibration signal 70 of FIG. 5.

Referring to FIG. 6, the control unit 84 may calculate the power spectra 122 by using the second peak value 91 of the pressure signals 90 in the second central region 92. For example, the power spectra 122 may be calculated by a Fourier transform method for a contraction period among the vibration signals 70. The power spectra 122 may be represented by a power value according to a change in frequency of vibration. According to an example, the power spectra 122 may include a first power spectrum 124, a tenth power spectrum 126, and a twentieth power spectrum 128. The first power spectrum 124 may be calculated based on the first vibration signal 75. The tenth power spectrum 126 may be calculated based on the tenth vibration signal 57. The twentieth power spectrum 128 may be calculated based on the twentieth vibration signal 79.

FIG. 7 shows an example of a first cumulative power 132 and a twentieth cumulative power 136 of the power spectra 122 of FIG. 6.

Referring to FIG. 7, the control unit 84 may obtain the first cumulative power 132 and the twentieth cumulative power 136 by using the power spectra 122. The first cumulative power 132 may be a cumulative power value of the first power spectrum 124. The twentieth cumulative power 136 may be a cumulative power value of the twentieth power spectrum 128. A cumulative power value may be normalized to have a maximum value of 1.

The first cumulative power 132 may have a first spectral edge frequency 30 value of about 15 Hz, and the twentieth cumulative power 136 may have a second spectral edge frequency 30 value of about 11 Hz. The first and second spectral edge frequency 30 values may be values in which cumulative power is about 0.3. The control unit 84 may calculate muscle fatigue by using the first and second spectral edge frequency 30 values of the first cumulative power 132 and the twentieth cumulative power 136. The muscle fatigue may be a percentage of a value obtained by dividing a difference between the first and twentieth spectral edge frequency 30 values by the first spectral edge frequency 30 value. When the first and twentieth spectral edge frequency 30 values are respectively 15 Hz and 11 Hz, the muscle fatigue may be calculated to be about 26.6%. A spectral edge frequency may vary from 10 to 90 depending on a target muscle, an experimental protocol, or the like.

Therefore, the wearable muscle activity measurement device 100 for diagnosing a muscle condition of the present invention may obtain muscle activity and muscle fatigue by using the mechanomyography sensors 30 and the force myography sensor 40, and may reduce and minimize noise of the muscle fatigue.

Although not illustrated, the wearable muscle activity measurement device 100 for diagnosing a muscle condition of the present invention may further include an inertial sensor (IMU) and an electromyogram (EMG) sensor mounted on the main substrate 10. The inertial sensor may detect or monitor a musculoskeletal motion and ab active muscle direction. The electromyogram sensor may detect a change in impedance according to the activity of a muscle.

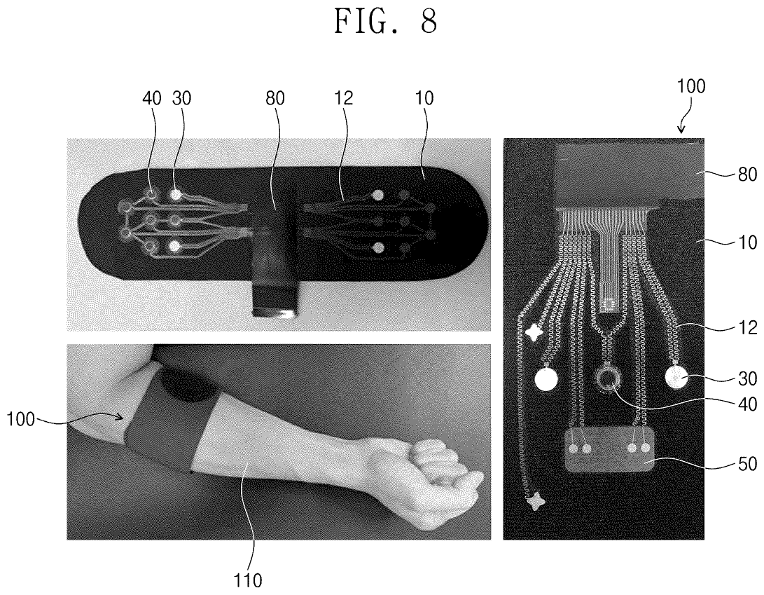

FIG. 8 shows an example of the wearable muscle activity measurement device 100 for diagnosing a muscle condition according to an embodiment of the inventive concept.

Referring to FIG. 8, the wearable muscle activity measurement device 100 for diagnosing a muscle condition of the present invention may be fastened to an arm 110 of the human body by using the main substrate 10 of a textile, such as a fiber, a film, or a thread. The electrode lines 12 may include stretchable lines having a serpentine shape. The electrode lines 12 may have a horseshoe shape, but the embodiment of the inventive concept is not limited thereto. The electrode lines 12 may have mechanical and electrical reliability with respect to the stretchability of a substrate.

The mechanomyography sensors 30, the force myography sensor 40, the motion sensor 50, and the communication module 80 may be configured in the same manner as in FIG. 1 and FIG. 2.

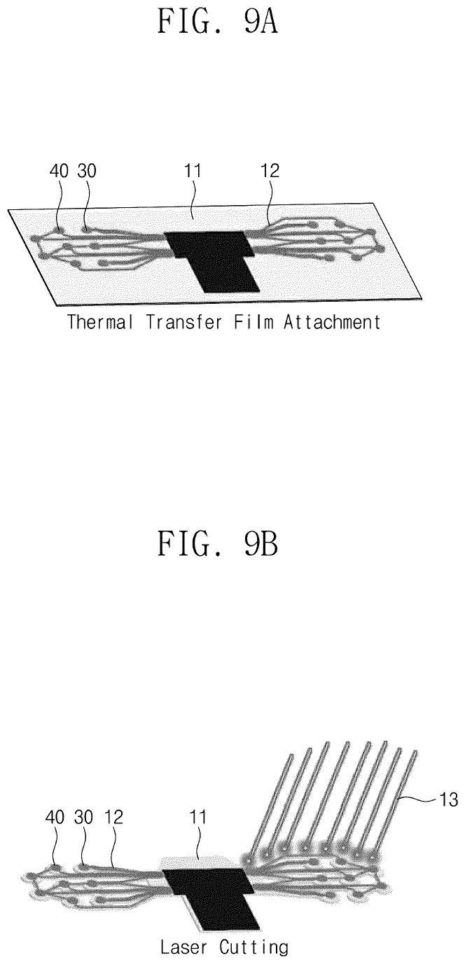

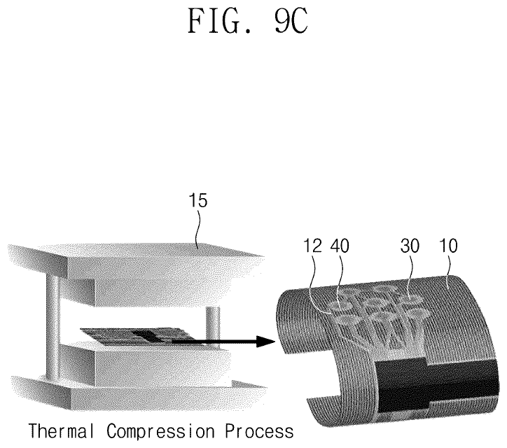

FIG. 9A to FIG. 9C show an example of a textile transfer process of the wearable muscle activity measurement device 100 for diagnosing a muscle condition of the present invention.

Referring to FIG. 9A to FIG. 9C, the mechanomyography sensors 30 and the force myography sensor 40 may be manufactured on a polyimide substrate (not shown), and then transferred to the main substrate 10 by means of a thermal transfer film 11. For example, the mechanomyography sensors 30 and the force myography sensor 40 may be printed and mounted on a polyimide substrate in the form of a multi-channel array.

Referring to FIG. 9A, the mechanomyography sensors 30 and the force myography sensor 40 may be attached to the thermal transfer film 11.

Referring to FIG. 9B, the thermal transfer film 11 may be cut by a laser cutting process. A laser beam 13 may cut the thermal transfer film 11 along edges of the electrode lines 12, the mechanomyography sensors 30, and the force myography sensor 40.

Referring to FIG. 9C, the thermal transfer film 11 may mount the electrode lines 12, the mechanomyography sensors 30, and the force myography sensor 40 on the main substrate 10 by a thermal compression process. A thermal compressor 15 may thermally compress the thermal transfer film 11 onto the main substrate 10.

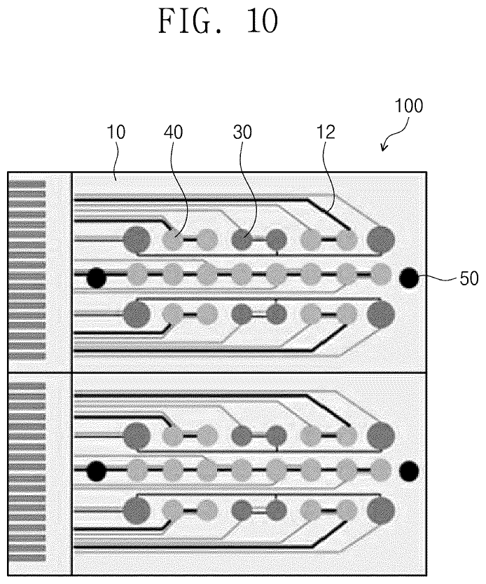

FIG. 10 shows an example of an array of the force myography sensor 40, the mechanomyography sensors 30, and the motion sensor 50 of FIG. 1 and FIG. 2.

Referring to FIG. 10, the mechanomyography sensors 30, the force myography sensor 40, and the motion sensors 50 may be arranged in an array form.

The mechanomyography sensors 30 and the force myography sensor 40 may be connected to the electrode lines 12 to be arranged in one direction. The mechanomyography sensors 30 may be larger or smaller than the force myography sensors 40. The force myography sensors 40 may be provided between the mechanomyography sensors 30. The motion sensor 50 may be provided on the main substrate 10 at both edges of the mechanomyography sensors 30 and the force myography sensors 40.

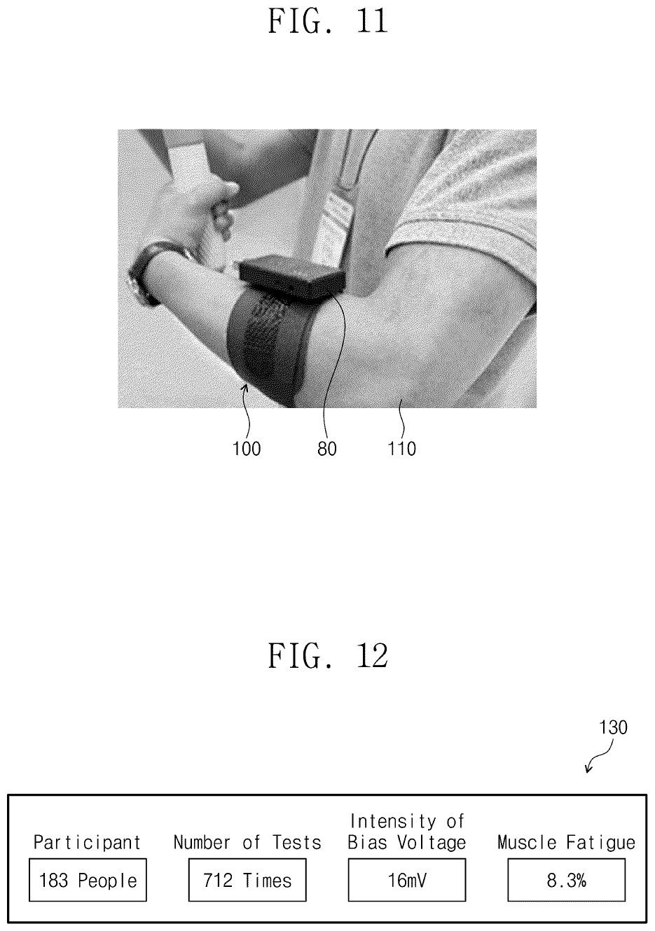

FIG. 11 shows an illustration of wearing the wearable muscle activity measurement device 100 for diagnosing a muscle condition of the present invention.

Referring to FIG. 11, the wearable muscle activity measurement device 100 for diagnosing a muscle condition of the present invention may be fastened to the arm 110 of the human body, so that the communication module 80 may be disposed in an upper portion of the arm 110.

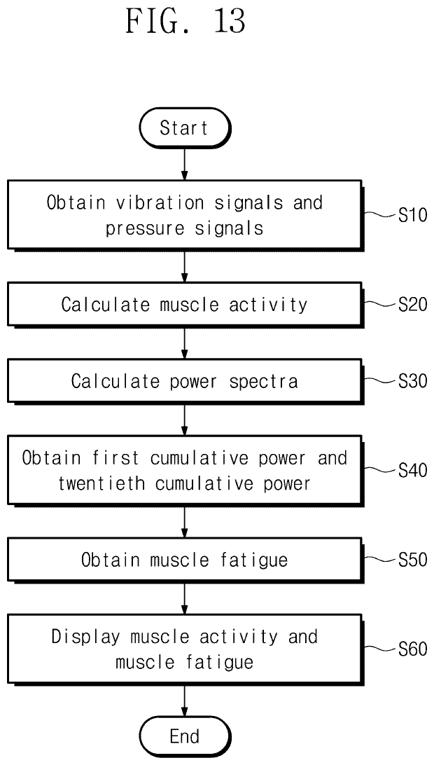

FIG. 12 shows an example of a display device 130 for displaying muscle activity statistical data measured from the wearable muscle activity measurement device 100 for diagnosing a muscle condition of the present invention.

Referring to FIG. 12, the control unit 84 may use the display device 130 to display a muscle activity evaluation participant, the number of tests, voltage intensity, and muscle fatigue to a user.

FIG. 13 shows an example of a method for measuring muscle activity of a wearable muscle activity measurement device 100 for diagnosing a muscle condition according to an embodiment of the inventive concept.

Referring to FIG. 13, a control unit 84 obtains vibration signals 70 and pressure signals 90 by using detection signals of mechanomyography sensors 30 and force myography sensors 40 S10. The pressure signals 90 may include a first pressure signal 95, a tenth pressure signal 97, and a twentieth pressure signal 99 overlapping in a first peak region 73. The vibration signals 70 may include a first vibration signal 75, a tenth vibration signal 57, and a twentieth vibration signal 79 overlapping in a second peak region 93.

Next, the control unit 84 obtains first peak values 71 of the pressure signals 90 of a first central region 72 to calculate muscle activity S20. The control unit 84 may calculate the muscle activity by using an average of the first peak values 71 of the first pressure signal 95, the tenth pressure signal 97, and the twentieth pressure signal 99 in the first central region 72.

Next, the control unit 84 obtains a second peak value 91 of the vibration signals 70 of a second central region 92 to calculate power spectra 122 S30. The control unit 84 may overlap the first peak region 73 of the pressure signals 90 and the second peak region 93 of the vibration signals 70 to remove a peak value of input signals of second edge regions 94 and a noise region 96, and may obtain the second peak value 91 of the pressure signals 90 in the second central region 92. The control unit 84 may convert the first vibration signal 75, the tenth vibration signal 57, and the twentieth vibration signal 79 by a Fourier transform method to obtain a first power spectrum 124, a tenth power spectrum 126, and a twentieth power spectrum 128 of the power spectra 122.

Thereafter, the control unit 84 uses the power spectra 122 to obtain a first cumulative power 132 and a twentieth cumulative power 136 S40. The first cumulative power 132 may have a first spectral edge frequency 30 value, and the twentieth cumulative power 136 may have a second spectral edge frequency 30 value.

Next, the control unit 84 uses the first spectrum edge frequency 30 value of the first cumulative power 132 and the second spectrum edge frequency 30 value of the twentieth accumulated power 136 to obtain muscle fatigue S50. The control unit 84 may calculate the muscle fatigue as a percentage of a value obtained by dividing a difference in value between the first spectral edge frequency 30 value and the second spectral edge frequency 30 value by the first spectral edge frequency 30 value.

Then, the control unit 84 displays the muscle activity and the muscle fatigue on the display device 130 S60.

Although not illustrated, the control unit 84 may analyze a signal pattern by using an analysis algorithm, learn a vibration signal 70 and a pressure signal 90 by using an artificial intelligence learning algorithm, predict a muscle condition, and output information on the muscle condition in a wired or wireless manner.

A wearable muscle activity measurement device for diagnosing a muscle condition according to an embodiment of the present invention may use a mechanomyography sensor and a force myography sensor to obtain muscle activity and muscle fatigue, and may reduce or minimize noise of the muscle fatigue.

Although the present invention has been described with reference to the accompanying drawings, it will be understood by those having ordinary skill in the art to which the present invention pertains that various changes in form and details may be made therein without departing from the spirit and scope of the present invention. Therefore, it is to be understood that the above-described embodiments are exemplary and non-limiting in every respect.

What is claimed is:

1. A wearable muscle activity measurement device for diagnosing a muscle condition, the device comprising:
a main substrate;
a communication module connected to the main substrate;
air pockets provided on one side of the main substrate adjacent to the communication module;
a motion sensor provided on the other side of the main substrate opposing the air pockets;
mechanomyography sensors provided on the center of the main substrate between the motion sensor and the air pockets to detect vibration signals; and
a force myography sensor provided on the main substrate between the mechanomyography sensors to detect pressure signals.

2. The device of claim 1, wherein the communication module comprises:
a module substrate; and
a control unit provided on the module substrate and connected to the motion sensor, the mechanomyography sensors, and the force myography sensor,
wherein the control unit overlaps a first peak region of the vibration signals and a second peak region of the pressure signals to distinguish noise regions of the pressure signals.

3. The device of claim 2, wherein the control unit uses a first peak value of first central regions between first edge regions of the vibration signals to calculate muscle activity.

4. The device of claim 3, wherein the control unit uses a second peak value of the pressure signals having a second central region overlapping the first central region to calculate muscle fatigue.

5. The device of claim 2, further comprising a pressure sensor provided on the module substrate or the air pockets.

6. The device of claim 2, further comprising a pressure sensor provided on the air pockets or the module substrate.

7. The device of claim 6, further comprising an air hose connecting the pressure sensor with the air pocket, which are on the module substrate.

8. The device of claim 1, wherein the force myography sensor comprises:
a first sensor substrate;
a plurality of first electrodes on the first sensor substrate;
a ring spacer provided on an edge of the first sensor substrate, which is an outer periphery of the first electrodes;
a pressure-sensitive structure provided on the ring spacer and the first electrodes; and
a passivation film provided on the pressure-sensitive structure.

9. The device of claim 1, wherein each of the above mechanomyography sensors comprises:
a second sensor substrate;
a housing case provided on an edge of the second sensor substrate;
second electrodes provided on the center of the second sensor substrate in the housing case;
a piezoelectric composite provided on the second electrodes;
a vibration transmission structure on the piezoelectric composite;
an elastic spring on the vibration transmission structure; and
a vibration transmission plate on the elastic spring.

10. The device of claim 1, wherein the main substrate comprises a textile substrate.

11. A method for measuring muscle activity, the method comprising:
obtaining vibration signals and pressure signals;
using the vibration signals and the pressure signals to calculate muscle activity;
using the pressure signals to calculate power spectra; and
using the power spectra to calculate muscle fatigue.

12. The method of claim 11, wherein the vibration signals comprise a first vibration signal, a tenth vibration signal, and a twentieth vibration signal, all of which have first peak values in a first peak region, wherein the first peak region includes a first central region and first edge regions on both sides of the first central region.

13. The method of claim 12, wherein the pressure signals comprise a first pressure signal, a tenth pressure signal, and a twentieth pressure signal, which have second peak values in a second peak region, wherein the second peak region includes a second central region overlapping the first central region, second edge regions overlapping the first edge regions, and a noise region at an outer periphery of the second edge regions.

14. The method of claim 13, wherein the power spectra comprise a first power spectrum, a tenth power spectrum, and a twentieth power spectrum, which are respectively obtained by Fourier transform of the first pressure signal, the tenth pressure signal, and the twentieth pressure signal.

15. The method of claim 14, further comprising using the first power spectrum and the twentieth power spectrum to obtain a first cumulative power having a first spectral edge frequency 30 value and a twentieth cumulative power having a second spectral edge frequency 30 value, wherein the muscle fatigue is calculated as a percentage of a value obtained by dividing a difference between the first spectral edge frequency 30 value and the second spectral edge frequency 30 value by the first spectral edge frequency 30 value.

16. A method for calculating muscle activity, the method comprising:
obtaining vibration signals including a first vibration signal, a tenth vibration signal, and a twentieth vibration signal overlapping in a first peak region, and pressure signals including a first pressure signal, a tenth pressure signal, and a twentieth pressure signal overlapping in second peak region;
integrating first peak values of the first vibration signal, the tenth vibration signal, and the twentieth vibration signal in a first central region of the first peak region to calculate muscle activity;
transforming the first pressure signal, the tenth pressure signal, and the twentieth pressure signal in a second central region of the second peak region by a Fourier transform method to calculate power spectra including a first power spectrum, a tenth power spectrum, and a twentieth power spectrum;
using the first power spectrum and the twentieth power spectrum to obtain a first cumulative power having a first spectral edge frequency 30 value and a twentieth cumulative power having a second spectral edge frequency 30 value; and
calculating a difference between the first spectral edge frequency 30 value and the second spectral edge frequency 30 value as muscle fatigue corresponding to a distribution rate of the first spectral edge frequency 30 value.

17. The method of claim 16, wherein the first central region and the second central region overlap each other.

18. The method of claim 16, wherein:
the first peak region further comprises a first edge region provided on both sides of the first central region; and
the second peak area further comprises a second edge region provided on both sides of the second central region and overlapping the first edge region.

19. The method of claim 18, wherein the second peak region further comprises a noise region at an outer periphery of the second edge region.

20. The method of claim 19, wherein the calculating of the power spectra comprises removing the second edge regions, and the first pressure signal, the tenth pressure signal, and the twentieth pressure signal in the noise region.
